(19) United States
(12) Patent Application Publication
(10) Pub. No.: US 2015/0257699 A1
(43) Pub. Date: Sep. 17, 2015

(54) MONITORING DEVICE FOR ANALYSING A SLEEP CONDITION

(71) Applicant: Deptracker ApS, Aarhus C (DK)

(72) Inventor: Stefan Bjerrum, Risskov (DK)

(21) Appl. No.: 14/433,302

(22) PCT Filed: Oct. 2, 2013
(86) PCT No.: PCT/EP2013/070528

§ 371 Date: Apr. 2, 2015

(30) Foreign Application Priority Data
Oct. 2, 2012 (EP) 12186967.1
Apr. 5, 2013 (EP) 13162466.0
May 30, 2013 (EP) 13169837.5

(51) Int. Cl. A61B 5/00

(52) U.S. Cl. CPC A61B 5/4815

(57) ABSTRACT

The invention provides a monitoring device for analysing a sleep condition of an individual. The device comprises at least a first motion sensor, a second motion sensor, and a processor. The first and second motion sensors are adapted to be positioned at an eyelid of the individual and to communicate a movement signal representing eye movement to the processor. The processor is adapted from the movement signal to provide an identifier significant for the sleep condition. The device may further comprise a tension sensor arranged to provide a tension signal based on sensed muscle tension in the eyelid. The motion sensors may comprises gyroscopes, accelerometers, or strain sensors.

### FIELD OF INVENTION

The present invention relates to a monitoring device for analysing a sleep condition.

### BACKGROUND

Different studies have revealed that poor sleep condition is one of the most important symptoms of depression. When the mental condition is improved, one of first indications is often an improved sleep condition. Likewise, can recurrence be identified by the return of poor sleep conditions.

Consequently, the monitoring of an individual to analyse a sleep condition may be seen as an important parameter in relation to depression. However, as the sleep conditions may also influence the individual in other ways not relating to mental health, the present invention is not limited to the use in relation hereto.

Traditionally, monitoring of sleep condition is carried out at a clinic where electrodes are attached to the head of the individual. This is expensive due to the apparatus, the number of electrodes, and the highly qualified staff needed for this procedure. Furthermore, different studies have revealed, that the fact that the studies are not suitable for being carried out at home, or are subject to uncertainty due to errors, and/or the fact that a number of electrodes have to be attached to the head of the individual, for a significant number of individuals influence the result of the monitoring, as the monitoring itself is experienced as unpleasant and insecure.

### DESCRIPTION OF THE INVENTION

It is an object of embodiments of the invention to provide an improved monitoring device for analysing a sleep condition.

It is a further object of embodiments of the invention to provide a monitoring device which can be used at home.

It is an even further object of embodiment of the invention to provide a monitoring device which can be used without the use of complicated equipment and without attaching a number of electrodes to the head of the individual who is monitored.

According to a first aspect, the invention provides a monitoring device for analysing a sleep condition of an individual, the device comprising at least a first motion sensor, a second motion sensor, and a processor, the first and second motion sensors being adapted to be positioned at an eyelid of the individual and to communicate a movement signal representing eye movement to the processor, and the processor being adapted, from the movement signal, to provide an identifier significant for the sleep condition.

As movement of the eyes of an individual may indicate the stage of sleep, the monitoring device is adapted to provide an identifier significant for a sleep condition based on monitoring of movement of the eyes.

The motion sensors may comprise gyroscopes and/or accelerometers and/or strain sensors. In one preferred embodiment of the invention, each of the motion sensors comprises a gyroscope. In another preferred embodiment, each of the motion sensors comprises an accelerometer. Any suitably sized accelerometer may be employed in the present invention, such as, e.g., that disclosed at http://micromachine.stanford.edu/˜kuanlinc/Professional/Mini %20Accelerometer.html as available on 3 Feb. 2013. Accelerometers and gyroscopes may suitable be implemented in accordance with the principles disclosed in H. J. Luinge et al.: “Measuring orientation of human body segments using miniature gyroscopes and accelerometers”, Medical & Biological Engineering & Computing 2005, Vol. 43, pp. 273-282. In a further preferred embodiment of the invention, each of the motion sensors comprises a strain sensor. By strain sensor should be understood a sensor capable of sensing deformation, such as tension. The strain sensor may be a piezo sensor, or a sensor based on crystals, fibres, liquid, etc.

The two motion sensors are preferably attached to a single eyelid of a patient. The motion sensors may be positioned on the upper eyelid or at the lower eyelid. Alternatively, one motion sensor may be positioned at the upper eyelid, while the second motion sensor is positioned on the corresponding lower eyelid. By monitoring movements by means of two motion sensors, their mutual movement or displacement may be determined, thereby resulting in a measure of the movement of the eyes. At least a first motion sensor and second motion sensor are adapted to be positioned at an eyelid of the individual. This may be done by the individual itself or by assistance of another person. Thus, the size of the motion sensors may be so small that the motion sensors themselves do not significantly interfere with the monitoring. If only a single motion sensor is used, it will not be possible to determine whether the monitored movement is caused by movement of the eye or the head of the individual. By use of two motion sensors, their mutual movement or displacement may be determined, thereby resulting in a measure of the movement of the eyes in which movement of the head has been eliminated.

The motion sensors may be adapted for wireless communication with the processor, thereby allowing the movement signal to be communicated to the processor without the use of wires which may interfere with the monitoring.

In contrast to traditional monitoring using electrodes attached to the head of the individual, motion sensors do not measure an electrical potential, and the monitoring according to the invention may therefore be less sensitive to electrical circuits or even completely independent on surrounding electrical signals.

The motion sensors sense movement of the eye behind the eyelid. This is possible as the eye is substantially round, i.e. circular/spherical, with the exception that the cornea is arranged at an outer side of the eye. When the eyes move, this movement may be seen at the eyelid due to movement of a bulge at the eyelid resulting from the cornea. This movement can be sensed by at least one of the first and second motion sensors. The sensed movement is communicated to the processor in the form of an electrical movement signal. The processor provides an identifier significant for the sleep condition based on this movement signal.

If both the first and second motion sensors sense movement, the processor may combine the movement signals, thus providing an identifier based sensed movement from both motion sensors.

Sleep may be divided into different stages; i.e. awake, shallow sleep, heavy sleep, and REM sleep (Rapid eye movement). As the different stages may be characterised by different levels of eye movements, the monitoring device can be used in different analyses of a sleep condition of an individual.

The below table includes different characteristics for the different stages relating to sleep.

| Stage | Eye movement | Muscle tension (upper eyelid) |
|---|---|---|
| Awake | No eye movement | Both vertical and horizontal |
| Shallow | Slow movements in the | Minimal tension both vertically |
| sleep 1 | form of eye rolling | and horizontally |
| Shallow | No eye movement | No vertical tension, minimal |
| sleep 2 | | horizontal tension |
| Heavy sleep | No eye movement | No vertical tension, horizontal |
| | | tension gradually stops |
| REM sleep | Rapid eye movements | No tension |

It may, as an example be possible to register: when an individual tries to go to sleep, how long time is takes to go to sleep, the length of shallow sleep period, the length of a heavy sleep period, the length of a REM sleep period, how many times the individual is awake during a monitoring period, e.g. during a night, etc.

As illustrated in the above table, there is no eye movement when an individual is awake, and is trying to go to sleep. However, when the individual has gone to sleep, the eyes will start to roll slowly from one side to the other.

To enable evaluation of the above mentioned parameters, e.g. to evaluate the speed of the eye movement, embodiments of the device according to the invention, in which the motion sensors are provided as gyroscopes and/or strain sensors, may comprise a timer or similar structure enabling the device to convert the movement signal into a speed or acceleration signal.

To facilitate attachment of the first and second motion sensors to the eyelid, the first and second motion sensors may be attached to a base. The base may have a lower surface facing towards the eyelid, and an opposite upper surface at which first and second motion sensors are attached. Thus, the first and second motion sensors may be attached to the eyelid substantially simultaneously.

To increase the area of the eyelid at which it may be possible to sense eye movements, it may be an advantage to arrange the first and second motion sensors at a distance from each other. As an example, the first motion sensor can be arranged at the eyelid toward the nose of the individual, whereas the second motion sensor can be arranged at the eyelid close toward the ear, or reverse.

Changes in the REM sleep, which can be registered by the use of the first and second motion sensors, may be seen as characteristic for depression. As an example, the REM sleep may occur earlier for individuals suffering from depression compared to individuals not suffering from depression.

It may however, be an advantage also to include a measure for when the individual goes to sleep. As an example, this may be monitored by use of a tension sensor.

Thus, the monitoring device may further comprise at least one tension sensor, such as a piezo sensor. The tension sensor may be adapted to be arranged on the eyelid and to provide a tension signal based on a sensed muscle tension in the eyelid. The tension sensor may be an independent sensor which can be positioned independently of the first and second motion sensors. Alternatively, the at least one tension sensor may be attached to the base at which the first and second motion sensors are attached.

In one embodiment, a first tension sensor of the at least one tension sensors is arranged to sense substantially vertical muscle tension and a second tension sensor of the at least one tension sensors is arranged to sense substantially horizontal muscle tension, thereby enabling differentiation between horizontal and vertical muscle tension.

If a tension sensor is positioned at the eyelid, when the eye is closed, the muscle is relaxed in the vertical direction, but in the horizontal direction, the muscle is tensed in order to keep the eye closed. This state can be seen as the basis for monitoring of muscle tension.

During shallow sleep, the eye will be kept closed and no vertical muscle tension will be registered. However, during shallow sleep the muscle will relax and the tension sensor sensing horizontal muscle tension will sense this relaxation.

When the individual wakes up, both tension sensors will sense muscle tension due to opening of the eye.

By use of at least two tension sensors, where the first tension sensor of the at least one tension sensors is arranged to sense substantially vertical muscle tension and the second tension sensor of the at least one tension sensors is arranged to sense substantially horizontal muscle tension, it may be possible to register: when the individual goes to sleep, when the heavy sleep occurred, if the individual has been awake during the monitoring period, the length of such an awake period, etc.

In an embodiment in which at least one of the motion sensors comprises a strain sensor, the first motion sensor may be constituted by the first tension sensor and/or the second motion sensor may be constituted by the second tension sensor, as the motion sensor(s) in this embodiment communicate a movement signal based on monitoring of tension.

By combining monitoring results from the first motion sensor, the second motion sensor, and the at least two tension sensors being adapted to sense horizontal and vertical muscle tension, respectively, numerous elements associated with sleep can be monitored.

The below table includes different elements and the associated characteristics.

| Element | Monitored characteristics |
|---|---|
| nascent tiredness | eye rolling, decreased vertical muscle |
| | tension which is registered by reduced |
| | deformation of one of the tension sensors |
| when an individual | no eye movement, no deformation of the |
| tries to go to sleep | tension sensor |
| how long time it | eye rolling, decreasing muscle tension, |
| takes before the | especially vertical, which is registered by |
| individual sleeps | deformation of the tension sensor |
| when the sleep occurred | significant decrease in vertical muscle |
| | tension |
| the length of the time | decreasing muscle tension, especially |
| period with shallow sleep | vertically, but also horizontally |
| when the heavy | further decreased muscle tension, |
| sleep occurred | especially horizontally, which is |
| | registered by increasing deformation of |
| | another tension sensor |
| the length of the time | no eye movement, very low muscle |
| period with heavy sleep | tension |
| when the REM sleep occurred | rapid eye movements start |
| the length of the time | rapid eye movements |
| period with REM sleep | |
| how many times the | increased muscle tension, opening of the |
| individual has been awake | eyes |
| during the monitoring period | |

Thus, the processor may be adapted to provide the identifier significant for the sleep condition based on the movement signal in combination with the tension signal.

As an alternative to the use of a tension sensor, a third motion sensor may be provided to facilitate registration of when the individual goes to sleep. The third motion sensor may be positioned at an arbitrary position of the head of the individual. When the head moves, all three motion sensors may be influenced substantial identically. However, when the individual has gone to sleep, movements of the eye will stop, and the registrations of the first and second motion sensors may be different from the registrations from the third motion sensor, thereby providing an indication of when the individual has gone to sleep.

The monitoring device may further comprise a data storage for storing at least one of the movement signal and the tension signal. Movement signals and/or tension signals may thus be stored during an entire monitoring period, thereby allowing for later data processing of the signals.

Data processing of at least one of the signal may be carried out by the monitoring device, at the monitoring device may further comprise a data processing unit for providing a processed signal based on at least one of the movement signal and the tension signal.

Furthermore, the monitoring device may comprise a transmitter which is adapted to transmit at least one of the movement signal, the tension signal, and the processed signal. The movement signal, the tension signal and/or the processed signal may be process to e.g. a sleep analyst, who may evaluate the transmitted signals and subsequently report back to the individual who has been monitored.

At least one of the motion sensors and the tension sensor may comprise a lower surface being at least partly adhesive to facilitate attachment to the eyelid. Alternatively, the lower surface of the base may be at least partly adhesive to facilitate attachment to the eyelid.

In order to protect the first and second motion sensors and a tension sensor, if present, the monitoring device may further comprise a cover being adapted to cover the motion sensors and the tension sensor.

In an alternative embodiment, the monitoring device comprises a strapping structure facilitating positioning of at least one of the motion sensors and the tension sensors at the eyelid when strapping the strapping structure to the individual. This may e.g. be achieved by attaching at least one of the motion sensors and the tension sensors to a mask-like structure which may be positioned in front of the eye of the individual whereupon the strapping structure may be tightened to ensure correct positioning of the motion sensor(s) and/or tension sensor(s) at the eyelid.

The mask-like structure may further comprise a distance adjustment structure facilitating movement of at least one of the motion sensors and the tension sensors relative to the eyelid. This enables correct positioning of the mask-like structure, including fastening of the strapping structure, before positioning the motion sensor(s) and/or tension sensor(s) at the eyelid. The distance adjustment structure may be operated manually, so that the individual himself/herself can adjust the distance and thereby position the motion sensor(s) and/or tension sensor(s) at the eyelid when ready to start the monitoring period. Alternatively, the adjustment may be carried out automatically in response to a closed-eye-signal, e.g. provided by a single tension sensor positioned at the eyelid. Thereby, it may be achieved that the motion sensor(s) and/or tension sensor(s) are positioned at the eyelid, when the eye closes and is removed again when the eye opens.

According to a second aspect, the invention provides a method of providing an identifier significant for a sleep condition, the method comprising the steps of:
providing a first motion sensor, a second motion sensor, and a processor;
positioning the first and second motion sensors at an eyelid of an individual;
providing a movement signal representing eye movement from at least one of

the motion sensors;
communicating the movement signal to the processor; and
using the processor to process the movement signal to provide the identifier.

It should be understood, that a skilled person would readily recognise that any feature described in combination with the first aspect of the invention could also be combined with the second aspect of the invention, and vice versa.

The monitoring device according to the first aspect of the invention is suitable for performing the method steps according to the second aspect of the invention. The remarks set forth above in relation to the monitoring device are therefore equally applicable in relation to the method.

The method may further comprise the steps of:
providing at least one tension sensor;
positioning the at least one tension sensor at the eyelid;
providing a tension signal representing sensed muscle tension from the at least one tension sensor;
communicating the tension signal to the processor; and
using the processor to process the movement signal and the tension signal in combination to provide the identifier.

Particularly, the method may be used for identifying symptoms of depression by use of the identifier. This is done by identifying poor sleep condition which is a symptom of depression.

It should be understood, that the steps of providing the motion sensors and the at least one tension sensor may be combined, and that the steps of positioning the motion sensors and the tension sensor(s) may likewise be combined, as the motion sensors and the tension sensor(s) may be built into a common unit, which may be arranged on the eyelid of the individual who will have his/her sleep condition analysed.

In a particular embodiment, the motion sensors are constituted by the tension sensors.

### BRIEF DESCRIPTION OF THE DRAWINGS

Embodiments of the invention will now be further described with references to the drawings, in which:

FIG. 1 illustrates a first motion sensor, a second motion sensor, and a tension sensor positioned at an eyelid,

FIGS. 2a-2c illustrate movement of the eye behind an eyelid at which two motion sensors are positioned,

FIG. 3 illustrates horizontal and vertical muscle tension,

FIGS. 4a-4c illustrate deformation of a tension sensor in response to horizontal and vertical muscle tension,

FIG. 5 illustrates an embodiment of a monitoring device comprising a first motion sensor and a second motion sensor,

FIG. 6 illustrates an embodiment of a monitoring device comprising a first and second motion sensor, and two tension sensors,

FIG. 7 illustrates an embodiment of a monitoring device attached to the eyelid of an individual,

FIG. 8 illustrates an embodiment of a monitoring device attached to the eyelid of an individual, the sensor comprising a cover,

FIGS. 9a-9c illustrate an alternative embodiment of a monitoring device,

FIGS. 10a-10b illustrate a part of a further alternative embodiment of a monitoring device,

FIG. 11 illustrates an example of monitored data, and

FIG. 12 illustrates experimental data for horizontal movement of an eye.

### DETAILED DESCRIPTION OF THE DRAWINGS

It should be understood that the detailed description and specific examples, while indicating embodiments of the invention, are given by way of illustration only, since various changes and modifications within the spirit and scope of the invention will become apparent to those skilled in the art from this detailed description.

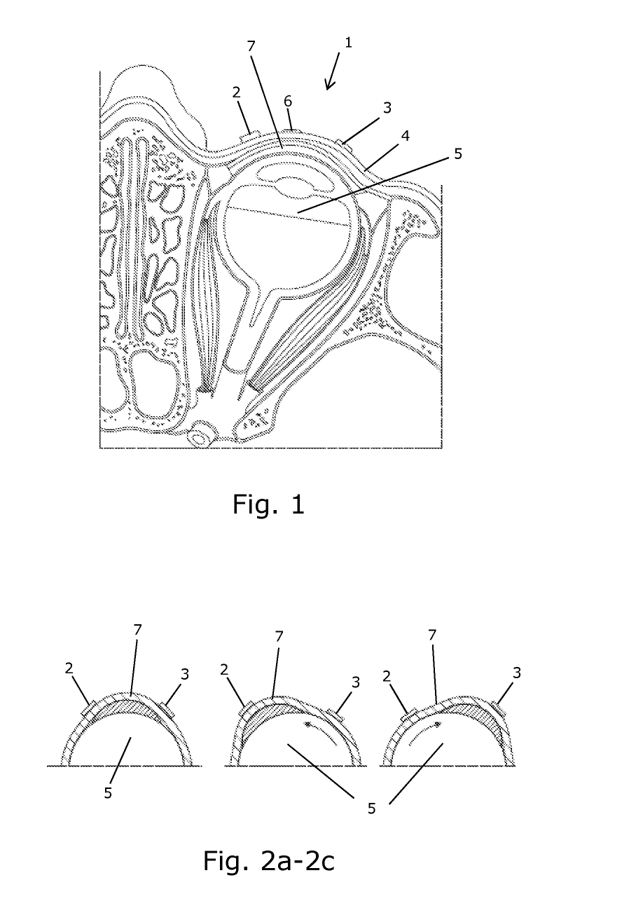

FIG. 1 illustrates elements of a monitoring device 1 for analysing a sleep condition of an individual. The illustrated embodiment of the monitoring device 1 comprises a first motion sensor 2, a second motion sensor 3, and a processor (not illustrated). The first and second motion sensors 2, 3 are adapted to be positioned at an eyelid 4 of the individual and to communicate a movement signal representing eye movement to the processor. The processor is adapted from the movement signal to provide an identifier significant for the sleep condition. In FIG. 1, which is a cross section of an eye 5, the first motion sensor 2, the second motion sensor 3, and a tension sensor 6 are positioned at the eyelid 4.

FIGS. 2a-2c illustrate movement of the eye 5 behind the eyelid 4 at which the first and second motion sensors 2, 3 are positioned. In FIG. 2a, the cornea 7 is centrally positioned, whereas FIGS. 2b and 2c illustrate the cornea 7 to the left and to the right, respectively. When comparing movement signals from the two motion sensors 2, 3, a measure for the movement of the eye 5 is achieved.

By providing a first motion sensor 2, such as a gyroscope or accelerometer, on the outer surface of the upper eyelid towards the patient's nose and a second motion sensor 3, such as a gyroscope or accelerometer, towards the canthus, an embodiment of a system is provided for determining eye movements. When the eye turns towards the canthus, that motion sensor, which is nearest to the canthus is lifted. The motion sensor nearest to the nose also moves, but its motion or displacement is smaller than the motion or displacement of the motion sensor nearest to the canthus. As all movements are determined by two motion sensors, and the respective signals are subtracted from each other, only the relative movement between the two sensors is being monitored; that relative movement provides a direct measure of the movement of the eye.

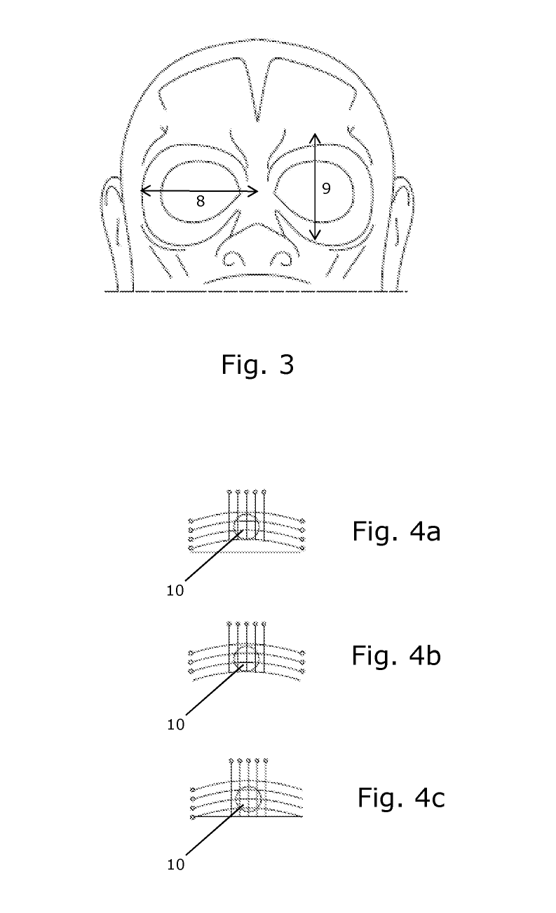

FIG. 3 illustrates horizontal and vertical muscle tension. The horizontal arrows 8 at the left eyelid 4′ illustrate horizontal muscle tension, whereas the vertical arrows 9 at the right eyelid 4″ illustrate vertical muscle tension. If a first tension sensor 10′ is arranged to sense substantially vertical muscle tension and a second tension sensor 10″ is arranged to sense substantially horizontal muscle tension, differentiation between horizontal and vertical muscle tension is enabled.

FIGS. 4a-4c illustrate deformation of a tension sensor 10, such as a piezo sensor, in response to horizontal and vertical muscle tension. The tension sensor 10 is adapted to be arranged on the eyelid 4 and to provide a tension signal based on a sensed muscle tension in the eyelid.

In FIG. 4a, the tension sensor 10 is positioned at a closed eyelid 4. The muscle is relaxed in the vertical direction, but in the horizontal direction, the muscle is tensed in order to keep the eye closed. This state can be seen as the basis for monitoring of muscle tension.

In FIG. 4b, the tension sensor 10 senses muscle tension both in the vertical and horizontal direction due to opening of the eye.

In FIG. 4c, the eye is closed during sleep and no vertical muscle tension is registered. However, during shallow sleep the muscle will relax and the tension sensor sensing horizontal muscle tension will sense this relaxation.

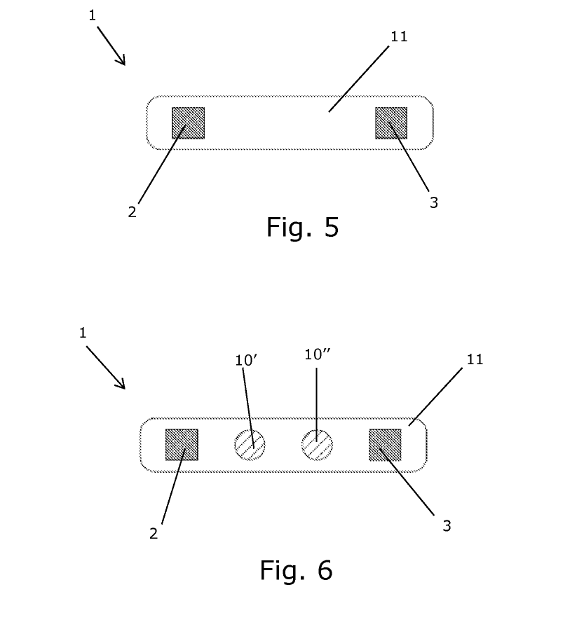

FIG. 5 illustrates an embodiment of a monitoring device 1 comprising a first motion sensor 2 and a second motion sensor 3. The first and second motion sensors 2, 3 are attached to a base 11. The base 11 has a lower surface facing towards the eyelid, and an opposite upper surface at which first and second motion sensors 2, 3 are attached. To facilitate positioning of the motion sensors, the lower surface of the base 11 is partly adhesive.

To increase the area of the eyelid at which it is possible to sense eye movements, the first and second motion sensors 2, 3 are arranged at a distance from each other.

FIG. 6 illustrates an embodiment of a monitoring device 1 comprising a first and second motion sensor 2, 3, and two tension sensors 10′, 10″. The first tension sensor 10′ is arranged to sense substantially vertical muscle tension, and the second tension sensor 10″ is arranged to sense substantially horizontal muscle tension.

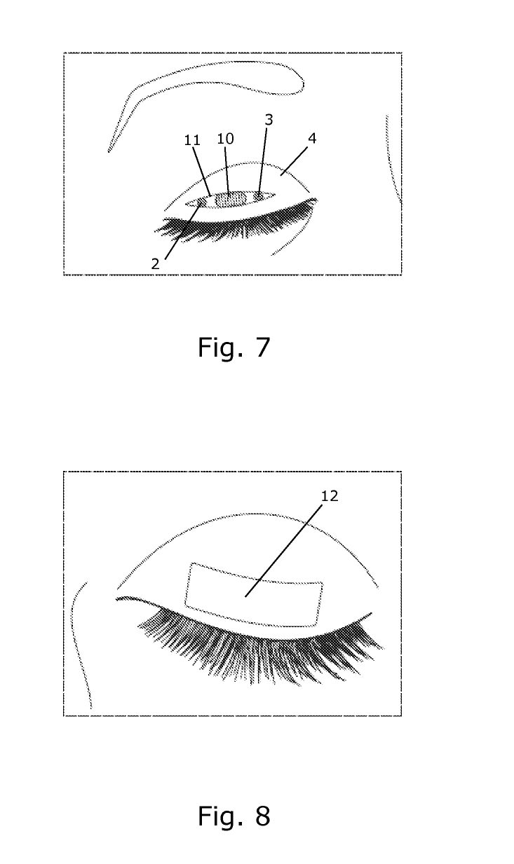

FIG. 7 illustrates an embodiment of a monitoring device 1 attached to the eyelid 4 of an individual. The monitoring device 1 comprises a first and second motion sensor 2, 3, and a tension sensor 10.

In FIG. 8, a cover 12 is arranged on top of the motion sensors and the tension sensor, thereby protecting them.

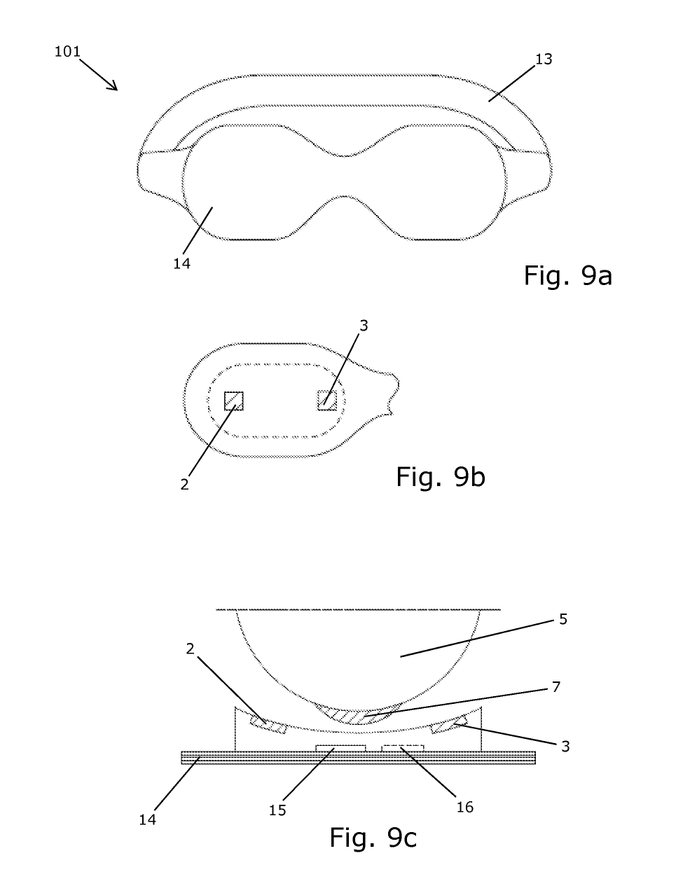

FIGS. 9a-9c illustrate an alternative embodiment of a monitoring device 101. The monitoring device 101 comprises a strapping structure 13 facilitating positioning the first and second motion sensor 2, 3 at the eyelid 4 when strapping the strapping structure 13 around the head (not shown) of the individual. This is achieved by attaching the motion sensors 2, 3 to a mask-like structure 14 which can be positioned in front of the eye 5 of the individual whereupon the strapping structure may be tightened to ensure correct positioning of the motion sensors 2, 3 at the eyelid 4. The monitoring device 101 further comprises a data processing unit 15 and a transmitter 16.

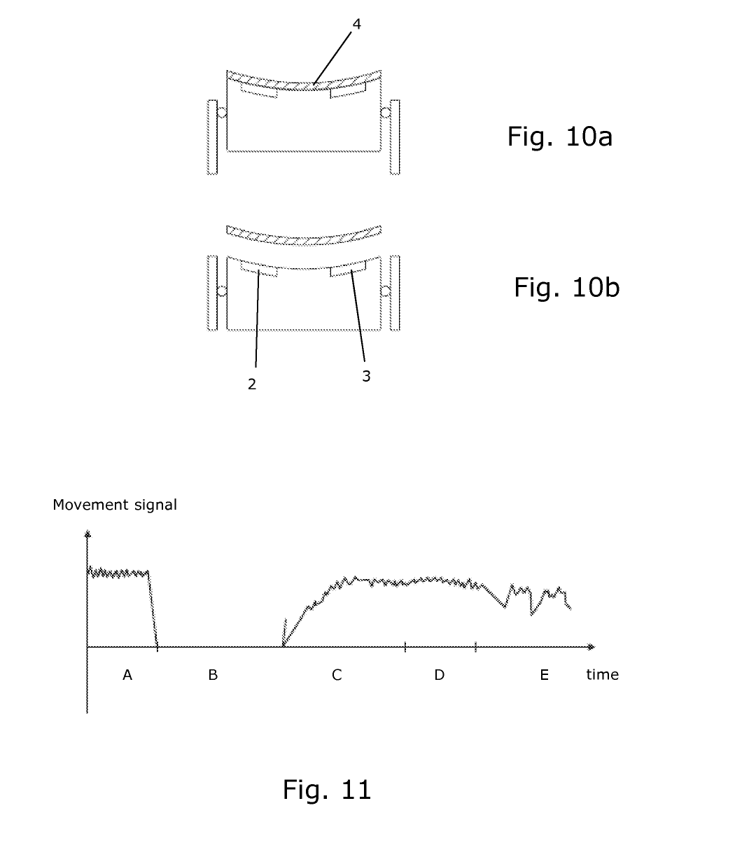

FIGS. 10a-10b illustrate a part of a further alternative embodiment of a monitoring device 101 which comprises a distance adjustment structure 16 facilitating movement of at least one of the motion sensors 2, 3 relative to the eyelid. This enables correct positioning of the mask-like structure (see FIGS. 9a-9c), including fastening of the strapping structure 13, before positioning the motion sensors 2, 3 at the eyelid.

The distance adjustment structure 16 may be operated manually, so that the individual can adjust the distance and thereby position the motion sensors at the eyelid 4 when ready to start the monitoring period. Alternatively, the adjustment may be carried out automatically in response to a closed-eye-signal, e.g. provided by a single tension sensor positioned at the eyelid.

FIG. 11 illustrates an example of monitored data. In the example, the movement signal is presented as a function of time. In the time period marked A, the individual is awake and has open eyes. In the time period marked B, the individual is awake and has closed eyes, i.e. no eye movements can be registered. In the time periods marked C, D, and E, the individual is sleeping. The time period C is a sleep period with shallow sleep, the time period D is a sleep period with heavy sleep, and the time period D is a sleep period with REM sleep.

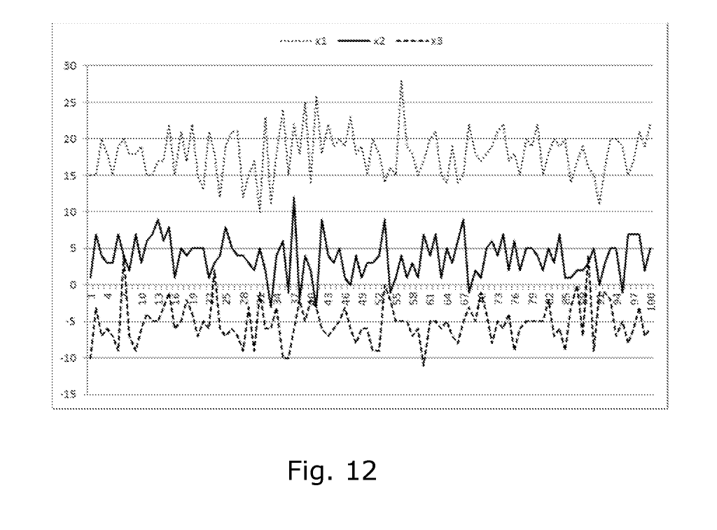

FIG. 12 illustrates horizontal movement of an eye, when the eye is looking towards the left side (x1), when the eye is looking straight forward (x2), and when the eye is looking towards the right side (x3), respectively. Along the x-axis, the different experiments are shown, i.e. a total of 100 experiments for each eye movement. The motion sensor, being a 6 g accelerometer has been positioned on the right side of the eyelid of the individual during the experiment.

### EXPERIMENTAL DATA

A monitoring device for analysing a sleep condition of an individual, wherein the device comprises accelerometers as motion sensors has been tested.

The accelerometers may be able to measure an angle in the range of 0-5 degrees, such as 0-4 degrees, such as 0-3 degrees, such as 0-2 degrees. The specific accelerometers used for obtaining experimental data are 6 g accelerometers which able to measure an angle in the range of 0-1.76 degrees.

In one experiment, one accelerometer was positioned on the right side of the eyelid of an individual, and the individual was asked to move his eye. A registration of horizontal movement of the eye was carried out by the accelerometer. In this experiment, only one motion sensor was used, as the individual was awake and was asked not to move his head.

FIG. 12 illustrates the horizontal movements of the eye, when the eye is looking towards the left side (x1), when the eye is looking straight forward (x2), and when the eye is looking towards the right side (x3). Along the x-axis, the different experiments are shown, i.e. a total of 100 experiments for each eye movement.

The average movement of the eye, when having an accelerometer positioned on the right side of the eyelid of an individual can be found in the below table.

| Average movement of the eye, | |
|---|---|
| when looking to the left | 18 |
| when looking straight forward | 0.06 |
| when looking to the right | −0.16 |

From the data in FIG. 12 and the average values displayed in the above table, it is clear that the measurements are significant, and that accelerometers can be used as motion sensor in a monitoring device according to the present invention.

If a more precise accelerometer, e.g. a 1 g accelerometer is applied, even more precise measurements are to be expected.

1. A monitoring device for analysing a sleep condition of an individual, the device comprising at least a first motion sensor, a second motion sensor, and a processor, the first and second motion sensors being configured to be positioned at a single eyelid of the individual and to communicate a movement signal representing eye movement to the processor, and the processor being configured, based on the movement signal, to provide an identifier significant for the sleep condition.

2. A monitoring device according to claim 1, wherein at least one of the first or second motion sensor comprises an accelerometer.

3. A monitoring device according to claim 1, wherein at least one of the first or second motion sensor comprises a gyroscope.

4. A monitoring device according to claim 1, wherein at least one of the first or second motion sensor comprises a strain sensor.

5. A monitoring device according to claim 2, comprising timer means enabling conversion of the movement signal to a speed or acceleration signal.

6. A monitoring device according to claim 1, wherein the first and second motion sensors are attached to a base, the base having a lower surface facing towards the eyelid, and an opposite upper surface at which first and second motion sensors are attached.

7. A monitoring device according to claim 1, wherein the first and second motion sensors are arranged at a distance from each other.

8. A monitoring device according to claim 1, comprising at least one tension sensor, the tension sensor being configured to be arranged on the eyelid and to provide a tension signal based on a sensed muscle tension in the eyelid.

9. A monitoring device according to claim 8, wherein a first tension sensor of the at least one tension sensors is arranged to sense substantially vertical muscle tension and a second tension sensor of the at least one tension sensors is arranged to sense substantially horizontal muscle tension.

10. A monitoring device according to claim 9, wherein at least one of the first motion sensor or the second motion sensor is constituted by a respective one of the first tension sensor or the second tension sensor.

11. A monitoring device according to claim 8, wherein the processor is configured to provide the identifier significant for the sleep condition based on the movement signal in combination with the tension signal.

12. A monitoring device according to claim 1, further comprising a data storage configured for storing at least one of the movement signal or the tension signal.

13. A monitoring device according to claim 11, further comprising a data processing unit configured for providing a processed signal based on at least one of the movement signal or the tension signal.

14. A monitoring device according to claim 13, further comprising a transmitter configured to transmit at least one of the movement signal, the tension signal, or the processed signal.

15. A monitoring device according to claim 1, wherein at least one of the motion sensors or the tension sensors comprises a lower surface being at least partly adhesive to facilitate attachment to the eyelid.

16. A monitoring device according to claim 1, further comprising a strapping structure configured to facilitate positioning of at least one of the motion sensors or the tension sensors at the eyelid when strapping the strapping structure to the individual.

17. A monitoring device according to claim 16, further comprising a distance adjustment structure configured to facilitate movement of at least one of the motion sensors or the tension sensors relative to the eyelid.

18. A method of providing an identifier significant for a sleep condition, the method comprising the steps of:
providing a first motion sensor, a second motion sensor, and a processor;
positioning the first and second motion sensors at a single eyelid of an individual;
providing a movement signal representing eye movement from the motion sensors including determination of mutual movement of the motion sensors;
communicating the movement signal to the processor; and
using the processor to process the movement signal to provide the identifier.

19. A method according to claim 18, further comprising the steps of:
providing at least one tension sensor;
positioning the at least one tension sensor at the eyelid;
providing a tension signal representing sensed muscle tension from the at least one tension sensor;
communicating the tension signal to the processor; and
using the processor to process the movement signal and the tension signal in combination to provide the identifier.

20. A method according to claim 18, comprising the step of identifying symptoms of depression by use of the identifier.
